(19) United States
(12) Patent Application Publication
(10) Pub. No.: US 2022/0308033 A1
(43) Pub. Date: Sep. 29, 2022

(54) AUTOMATIC MACHINE-GENERATED FOOD TASTE ANALYSIS AND LABELING

(71) Applicant: LINX SYSTEMS, INC., Bloomington, IL (US)

(72) Inventor: Andrew O'Neall, Bloomington, IL (US)

(21) Appl. No.: 17/702,176

(22) Filed: Mar. 23, 2022

Related U.S. Application Data
(60) Provisional application No. 63/165,648, filed on Mar. 24, 2021.

(51) Int. Cl. G01N 33/02; G06Q 50/02; G01N 1/28

(52) U.S. Cl. CPC G01N 33/025; G06Q 50/02; G01N 1/286

(57) ABSTRACT

One embodiment relates to a system that includes a modular testing attachment comprising at least one of a crusher attachment, a squeezer attachment, and a blender attachment, the modular testing attachment configured to obtain a substance from food or crop products, a universal attachment coupled to the modular testing attachment and structured to receive and advance the substance from the food or crop products, a multispectral tester coupled to the universal attachment and structured to receive the substance from the universal attachment and test the substance to output scientific values, and a computing system operably coupled to the modular testing attachment and the multispectral tester and configured to selectively or automatically operate components thereof

### BENEFIT CLAIM

This application claims the benefit under 35 U.S.C. §119(e) of provisional application 63/165,648, filed Mar. 24, 2021, the entire contents of which are hereby incorporated by reference for all purposes as if fully set forth herein.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright or rights whatsoever. © 2020-2021 Linx Systems, Inc.

### TECHNICAL FIELD

One technical field of the present disclosure is machine analysis of sensory attributes of food. Another technical field is automatic machine calculation of taste attributes of food, including automatic label printing.

### BACKGROUND

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

A variety of foods that are produced using modern agricultural techniques exhibit wide variance in human perceived taste, even when each food item is genetically identical to another. Examples include citrus fruits, strawberries, tomatoes, and grapes. Any individual orange, even from a recognized varietal such as Valencia, may vary widely in taste characteristics such as sweetness and acidity depending upon field conditions such as soil, rainfall, sunlight, and even position of the fruit on the tree.

Chemistry-based tests for selected taste characteristics are known; for example, pH and Brix can be measured in citrus juice. In conventional testing for these attributes, measurements and samples are taken from groves or blocks of farms. Testing typically occurs once or twice per growing season because manual selection of fruit, followed by laboratory tests at a remote location, involve large amounts of labor and other costs. Consequently, testing cannot be done more frequently throughout the year.

Additionally, current methods of testing may only be available for certain crops and not available for all crops. As such, only a fraction of all crops is being tested.

Furthermore, testing does not yield data that is useful to a retail consumer. For example, for oranges, some consumers prefer intense sweetness and others value acidity, yet the consumer has no way to know the true likely taste of a particular orange until it is purchased and consumed, unless the merchant incurs the cost of offering free samples at the point of sale, which is not always done. At retail locations such as food markets, it would be valuable for a consumer to know the specific taste characteristics of a lot of food before a purchase. However, current testing of food crops primarily focuses on size and appearance, and there is no practical way to rapidly score food items for taste attributes and to associate test results with data that is meaningful to consumers.

### SUMMARY

Mobile, computer-controlled testing apparatus is disclosed for use in field testing food or crop items to obtain scientific data values for a plurality of food attributes that impact human taste perception, such as acid and Brix, as well as ratio, weight, size, and color. The apparatus may incorporate multiple different testing attachments that are coupled to an attachment receiver, enabling modular connection of different attachments to conduct juicing, crushing, or other operations to yield testable food samples. The apparatus comprises a multispectral tester. Under stored program control, two or more of the scientific data values are transformed into two or more consumer score values based on one of a plurality of different, digitally stored scaled mappings of scientific data values to consumer score values. In the mappings, scaling and mapping correlations vary based upon the species and variety of food item. The two or more consumer score values are blended or weighted to yield a final consumer score value representing a taste of the food item. Consequently, a single score value can represent a variety of physical food characteristics that impact food taste and can serve as a reference to assist growers, wholesalers, retailers, or consumers to select crops or food having desired taste attributes, based upon objective tested values at multiple points in a field or farm.

One embodiment relates to a system that includes a modular testing attachment comprising at least one of a crusher attachment, a squeezer attachment, and a blender attachment, the modular testing attachment configured to obtain a substance from food or crop products, a universal attachment coupled to the modular testing attachment and structured to receive and advance the substance from the food or crop products, a multispectral tester coupled to the universal attachment and structured to receive the substance from the universal attachment and test the substance to output scientific values, and a computing system operably coupled to the modular testing attachment and the multispectral tester and configured to selectively or automatically operate components thereof. In some embodiments, the multispectral tester includes at least one of a refractometer, a mass spectrometer, an infrared camera, a visible light camera, a thermometer, modular bays, and a digital scale for mass measurements. In some embodiments, the universal attachment comprising a vacuum mechanism structured to advance the substance from the modular testing attachment to the multispectral tester. Alternatively or additionally, the universal attachment may be coupled to multiple testing attachments and the computing system configured to determine a type of food or crop product that is being tested, and automatically select and activate one of the multiple testing attachments that correspond to the type of food or crop product that is being tested.

In some embodiments, the computing system is configured to access baseline data from a database to process testing data for the substance and scale or weight the scientific values based on the baseline data. The system may also include a network interface coupled to the computing system, where the computing system is configured to generate human-readable scores based on the scientific values, and store the human-readable scores and the scientific values within the database. The human-readable scores may be related to at least one of sweetness, balance, weight, size, color, and bitterness.

In various embodiments, the system may include a fluid tank structured to provide a fluid comprising at least one of distilled water, isopropyl alcohol, and disinfectant to flush out the multispectral tester automatically after testing the substance. In various embodiments, the system may include two or more multispectral testers, each structured to receive at least a portion of the substance from the universal attachment, the computing system configured to determine a type of food or product being tested and selectively activate one or more of the multispectral testers based on the type of food or product being tested. Alternatively or additionally, the system may also include a user-interface screen coupled to the computing system configured to allow a user to select the type of food or product being tested.

Another embodiments relates to a computer-implemented method of testing food items. The method including obtaining scientific data values for one or more conventional attributes of a food item, transforming each of the scientific data values into two or more consumer score values using a digitally stored scaled mapping of scientific data values to consumer score values, forming a final consumer score value by combining and weighting the two or more consumer score values, and outputting the final consumer score value, wherein the final consumer score value represents a taste archetype for the food item. In some embodiments, the method may include generating label data comprising an encoded representation of the final consumer score value and printing a label comprising the encoded representation of the final consumer score value. In various embodiments, the method may include obtaining the scientific values by activating a mobile testing system structured to receive the food item, process the food item, and test the food item based on a type of the food item.

In some embodiments, the method may also include receiving, via a user interface, a selection of the type of the food item, activating one or more modular testing attachments of the mobile testing system, the one or more modular testing attachments structured to obtain a substance from the food item, and generating the scientific data values using one or more multispectral tester devices that receive the substance. The scientific data values may represent scores related to at least one of a pH scale, a Brix scale, a weight, and a color of the food item. The consumer scale values may indicate at least one of sweetness, balance, weight, size, color, and bitterness.

In another embodiment relates to a system having a mobile testing system configured to receive a food item and generate scientific data values for one or more conventional attributes of the food item one or more processors coupled to the mobile testing system, a memory device coupled to one or more processors. The mobile testing system includes a modular testing attachment structured to obtain a substance from the food item, and a multispectral tester structured to receive the substance and test the substance to generate scientific data values. Moreover, the memory device is structured to store instructions that, when executed by the one or more processors, cause the operations of, obtaining scientific data values from the mobile testing system, transforming each of the scientific data values into two or more consumer score values using a digitally stored scaled mapping of scientific data values to consumer score values, forming a final consumer score value by combining and weighting the two or more consumer score values, and outputting the final consumer score value, wherein the final consumer score value represents a physical characteristic of the food item. In some embodiments, the operations further include generating label data comprising an encoded representation of the final consumer score value and printing a label comprising the encoded representation of the final consumer score value. In various embodiments, the scaled mapping of scientific data values to consumer score values may be dependent on a type of the food item. In some embodiments, the system may also include a user interface coupled to the one or more processors, the user interface configured to provide a plurality of selections to a user, each of the plurality of selections being related to a type of food items.

Additionally, the appended claims may serve as a summary of the invention.

### BRIEF DESCRIPTION OF THE DRAWINGS

In the drawings:

FIG. 1 illustrates a distributed computer system showing the context of use and principal functional elements with which one embodiment could be implemented.

FIG. 2 illustrates an example computer-implemented process for deriving score values.

FIG. 3 illustrates an example computer-implemented process for compiling data for a taste database.

FIG. 4 illustrates an example computer-implemented process for generating a taste archetype.

FIG. 5, FIG. 6 illustrate examples of computing systems with which embodiments could be implemented.

### DETAILED DESCRIPTION

In the following description, for the purposes of explanation, numerous specific details are set forth in order to provide a thorough understanding of the present invention. It will be apparent, however, that the present invention may be practiced without these specific details. In other instances, well-known structures and devices are shown in block diagram form in order to avoid unnecessarily obscuring the present invention.

The text of this disclosure, in combination with the drawing figures, is intended to state in prose the algorithms that are necessary to program a computer to implement the claimed inventions, at the same level of detail that is used by people of skill in the arts to which this disclosure pertains to communicate with one another concerning functions to be programmed, inputs, transformations, outputs and other aspects of programming. That is, the level of detail set forth in this disclosure is the same level of detail that persons of skill in the art normally use to communicate with one another to express algorithms to be programmed or the structure and function of programs to implement the inventions claimed herein.

Embodiments are described in sections below according to the following outline:
1. General Overview
2. Structural & Functional Overview
3. Implementation Example — Hardware Overview

### 1. General Overview

A mobile, computer-controlled testing apparatus is disclosed for use in field testing food or crop items to obtain scientific data values for a plurality of food attributes that impact human taste perception, such as acid and Brix, as well as ratio, weight, size, and color. The apparatus may incorporate multiple different testing attachments that are coupled to an attachment receiver, enabling modular connection of different attachments to conduct juicing, crushing, or other operations to yield testable food samples. The apparatus comprises a multispectral tester. Under stored program control, two or more of the scientific data values are transformed into two or more consumer score values based on one of a plurality of different, digitally stored scaled mappings of scientific data values to consumer score values. In the mappings, scaling and mapping correlations vary based upon the species and variety of food item. The two or more consumer score values are blended or weighted to yield a final consumer score value representing a taste of the food item. Consequently, a single score value can represent a variety of physical food characteristics that impact food taste and can serve as a reference to assist growers, wholesalers, retailers, or consumers to select crops or food having desired taste attributes, based upon objective tested values at multiple points in a field or farm.

In particular embodiments, a digital electronic mobile testing system for food or crop items is disclosed. The mobile testing system may be deployed in various settings of a farm or field to test any of a plurality of different crops. As an example, and not by way of limitation, the mobile testing system may be deployed in a citrus farm or a strawberry farm, in a grove, block, or other unit. In particular embodiments, the mobile testing system may be configured to couple to a vehicle for mobility. As an example, and not by way of limitation, the mobile testing system may be configured to be placed in a bed of a truck, a trunk of a car, on the deck of an ATV, affixed to the implement interface of a tractor, or other locations.

In particular embodiments, the mobile testing system may be configured to instruct one or more users to perform a sequence of testing for a particular item under stored program control. As an example, and not by way of limitation, to completely test a grove of oranges, the mobile testing system may instruct the user to test a specified number of oranges from a specified number of trees in a particular block of a specified grove. As another example, the mobile testing system may instruct the user to test a specified number of strawberries plants in a specified block in a field. Different crops may require different testing guidelines or instructions to generate a complete testing profile of the crop. For instance, one crop may vary widely throughout a grove and another crop may be more consistent throughout a grove. The determination of testing instructions may be based on previously tested data and/or input from a grower or an expert.

In particular embodiments, the mobile testing system may include a universal attachment receiver to receive a modular testing attachment. As an example, and not by way of limitation, the universal attachment receiver may receive one or more of a crusher attachment, a squeezer attachment, or a blender attachment. In particular embodiments, the universal attachment receiver may use a combination of one or more attachments to extract a testable substance, such as juice, pulp, or a slurry or mash, from a food or crop item. As an example, and not by way of limitation, the mobile testing system may comprise a universal attachment to receive a squeezer to squeeze oranges to extract orange juice to test.

The mobile testing system may comprise a coupler to connect the universal attachment receiver to a multispectral tester and to route the substance extracted from the food or crop to be tested. In particular embodiments, the multispectral tester may include one or more of a refractometer, a mass spectrometer, infrared or visible light cameras, a thermometer, modular bays, and/or a digital scale for mass measurements.

In particular embodiments, the mobile testing system may include an ejector apparatus that is configured to dispose of previously tested substances. As an example, and not by way of limitation, after each test, the mobile testing system may be configured to eject the tested material automatically, or in response to manual selection of an eject button or other control. In particular embodiments, the mobile testing system may rely on the volume of a new substance to flush or remove previously tested substances out of the multispectral tester. As an example, and not by way of limitation, the mobile testing system may be configured to receive enough food or crop to generate enough substance to overcome trace amounts of the previously tested substance that may remain in the apparatus after a previous test. In particular embodiments, the mobile testing system may be coupled to a fluid tank that contains a fluid such as distilled water, isopropyl alcohol, or other fluid to flush out the multispectral tester between tests.

### 2. Structural & Functional Overview

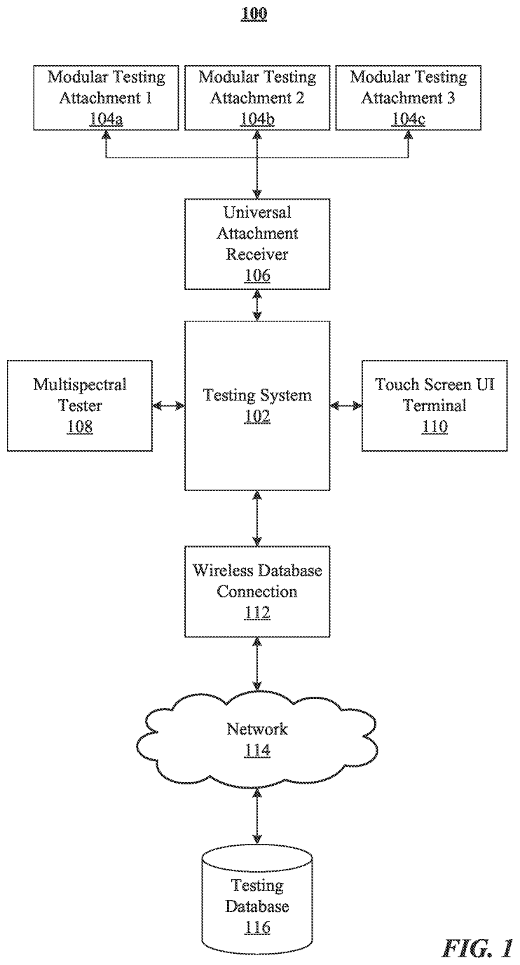

FIG. 1 illustrates a distributed computer system showing the context of use and principal functional elements with which one embodiment could be implemented.

In an embodiment, a distributed computer system 100 comprises components that are implemented at least partially by hardware at one or more computing devices, such as one or more hardware processors executing stored program instructions stored in one or more memories for performing the functions that are described herein. In other words, all functions described herein are intended to indicate operations that are performed using programming in a special-purpose computer or general-purpose computer, in various embodiments. FIG. 1 illustrates only one of many possible arrangements of components configured to execute the programming described herein. Other arrangements may include fewer or different components, and the division of work between the components may vary depending on the arrangement.

FIG. 1, and the other drawing figures and all of the description and claims in this disclosure, are intended to present, disclose and claim a technical system and technical methods in which specially programmed computers, using a special-purpose distributed computer system design, execute functions that have not been available before to provide a practical application of computing technology to the problem of machine learning model development, validation, and deployment. In this manner, the disclosure presents a technical solution to a technical problem, and any interpretation of the disclosure or claims to cover any judicial exception to patent eligibility, such as an abstract idea, mental process, method of organizing human activity or mathematical algorithm, has no support in this disclosure and is erroneous.

In particular embodiments, FIG. 1 illustrates a computer system 100 comprising a testing system 102, network 114 and testing database 116. The mobile testing system 102 couples to a universal attachment receiver 106, which is configured to couple to a plurality of modular testing attachments 104a-104c. For purposes of illustrating a clear example, three (3) modular testing attachments 104a-104c are shown in FIG. 1, but other embodiments may have fewer or more attachments.

The mobile testing system 102 may comprise a multispectral tester 108, a touch screen UI terminal 110, and a wireless database connection 112. Testing system 102 may be mechanically and electrically coupled to receiver 106, and electrically coupled to tester 108, terminal 110, and connection 112 via one or more digital electronic interfaces.

In particular embodiments, the wireless database connection 112 may be wirelessly coupled via a network 114 to a testing database 116. Access to the testing database 116 may be managed using a server computer or other host computer that is coupled to network 114.

In particular embodiments, as with the attachments, while a number of components of the computer system 100 are shown, other embodiments may include a different number of components. As an example, and not by way of limitation, the mobile testing system 102 may include multiple multispectral testers 108 or may be connected to multiple testing databases 116. In particular embodiments, while a certain configuration of the system 100 is shown, other embodiments may have a different configuration of components. As an example, and not by way of limitation, the mobile testing system 102 may combine the universal attachment receiver 106 directly with a modular testing attachment 104a, 104b, 104c.

The modular testing attachments 104a-104c may include only a squeezer attachment as the modular testing attachment 104a. As another example and not by way of limitation, the mobile testing system 102 may include both a squeezer attachment as the modular attachment 104a and a blender attachment as the modular attachment 104b. As an example, and not by way of limitation, the modular testing attachments 104a, 104b, 104c may include one or more of a crusher, a squeezer, a blender, and/or any other type of attachment to obtain a substance from food or crop. In particular embodiments, one or more modular testing attachments 104a, 104b, 104c may be detachable and changeable based on the food or crop that is being tested. Testing system 102 may include a computing device operating under stored program control and interoperating with terminal 110 to receive input to modify the configuration and use of attachments 104a, 104b, 104c and/or receiver 106. As an example, and not by way of limitation, a user may input, using terminal 110, a type of crop that is being tested and the mobile testing system 102 may configure itself to test for that type of crop by selecting or activating the correct one of the attachments 104a, 104b, 104c. For instance, if a user selects the crop to be tested is a strawberry, the mobile testing system 102 may be configured to select to equip a blender attachment in response to the selection.

In particular embodiments, the universal attachment receiver 106 may be configured to receive a substance from one or more of the modular testing attachments 104a, 104b, 104c. As an example, and not by way of limitation, universal attachment receiver 106 may be configured to couple to a squeezer attachment to receive juice from an orange to be tested. In particular embodiments, the universal attachment receiver 106 may include a vacuum mechanism to advance the extracted substance to the testing system 102. In particular embodiments, the universal attachment receiver 106 may be coupled to a water reservoir to flush out previously tested substances. As an example, and not by way of limitation, the universal attachment receiver 106 may operate under program control to draw or pump a fluid from a fluid reservoir to flush the multispectral tester 108. The universal attachment receiver 106 may be configured to redirect a fluid from a fluid reservoir to the multispectral tester 108. In particular embodiments, the universal attachment receiver 106 may be mechanically coupled to the testing system 102 using electrically operable values which may operate under program control to redirect the substance received from the universal attachment receiver 106 to the multispectral tester 108.

The testing system 102 may be coupled to the universal attachment receiver 106 to receive a substance/juice to be tested. The testing system 102 may be coupled to the multispectral tester 108, which the testing system 102 may direct the received substance/juice from the universal attachment receiver 106 to be tested by the multispectral tester 108. In particular embodiments, the testing system 102 comprises the computing device, noted above, to execute, under stored program control, the functions of the system 100 that are further described herein. The testing system 102 may be coupled to a touch screen UI terminal 110 to receive input signals specifying a selection of which food or crop is being tested. In response, a stored program executed using the computing device may select the correct attachment and/or drive the attachment to conduct specified operations such as squeezing, crushing, extracting, and initiating testing via tester 108.

In particular embodiments, the testing system 102 may be physically coupled or wirelessly coupled to a wireless database connection 112 to communicate with a testing database 116 via network 114. Once the testing system 102 receives data from multispectral tester 108, the testing system 102 may be programmed to store the data locally and/or send data to the testing database 116 via the wireless database connection 112. In particular embodiments, the testing system 102 may be programmed to process the data received from the multispectral tester 108 to categorize the data to be stored on the testing database 116. As an example, and not by way of limitation, the testing system 102 may be programmed to receive data from the multispectral tester 108 for an orange that is being tested and the testing system 102 may be programmed to determine whether the orange is sweet based on Brix values of the tested sub stance.

While the data processing is described as being performed by the testing system 102, testing database 116 and/or a host computer to which the testing database is coupled may be programmed to execute further testing or scoring operations. In particular embodiments, the testing system 102 may be coupled to a global positioning system (GPS) module and may be programmed to read the GPS module to obtain location data representing a then-current geographical location of the testing system. The testing system 102 may be programmed to send the location data in association with testing data of food or crop items, and to instruct database 116 to store the location data in association with the testing data.

In particular embodiments, the testing system 102 may be programmed to access data from testing database 116 and may be programmed to compare the testing data to stored data to derive scores for the tested substance. In particular embodiments, the testing system 102 may be programmed to access baseline data from the testing database 116 to process the testing data for a food or crop and may be programmed to scale or weight raw data values from tester 108 using the baseline data. As an example, and not by way of limitation, the testing system 102 may receive input that the food or crop being tested is an orange and may access baseline data for oranges from the testing database 116. In particular embodiments, the testing system 102 may use the baseline data to weight or refine the testing of the orange that was done by the multispectral tester 108.

In particular embodiments, the multispectral tester 108 may receive a substance to be tested from the testing system 102. In particular embodiments, the multispectral tester 108 may comprise one or more different testing devices that are structured or programmed to perform different tests on the substance. In particular embodiments, the multispectral tester may include one or more of a refractometer, a mass spectrometer, one or more infrared or visible light cameras, thermometer, digital scale, and other testing devices.

In particular embodiments, the multispectral tester 108 may include a transparent tube that may hold a tested substance to perform tests using the testing devices. As an example, and not by way of limitation, the multispectral tester 108 may use a transparent tube to hold orange juice or crushed strawberry pulp while performing testing using a refractometer. In particular embodiments, the multispectral tester 108 may use the digital scale to measure the mass or weight of the substance. In particular embodiments, the multispectral tester 108 may use the thermometer to measure a temperature of the substance being tested. In particular embodiments, the multispectral tester 108 may use cameras to identify a color of the substance being tested. The color of a substance may be used to identify imperfections of the substance or may correlate to known taste characteristics of a food item. In particular embodiments, the multispectral tester 108 may use the refractometer to measure a Brix value of the substance. In particular embodiments, the multispectral tester 108 may use the mass spectrometer to determine a spectral distribution of the substance. In particular embodiments, the multispectral tester 108 may generate data for physical and chemical properties of the tested substance.

In particular embodiments, the testing system 102 may be programmed to send instructions to the multispectral tester 108 to test for a specific food or crop. Each food or crop may have its own individual testing procedure that is programmed in software that the computing device of system 102 executes. For instance, a specific camera may be activated or turned on to perform a test on oranges (e.g., or substances obtained from oranges), but not on blueberries.

In particular embodiments, the touch screen UI terminal 110 may provide a text, graphical, or other visual interface with which a user may interact to initiate testing for a food or crop. In particular embodiments, the computing device of testing system 102 may be programmed to drive the touch screen UI terminal 110 to present an interface for a user to select what food or crop is being tested and/or a species, hybrid, variety, or varietal of a food or crop that is being tested. In particular embodiments, the computing device of testing system 102 may be programmed to drive the touch screen UI terminal 110 to present one or more tests that are being performed, or capable of being performed, on the food or crop. In particular embodiments, the computing device of testing system 102 may be programmed to drive the touch screen UI terminal 110 to allow the user to modify the testing performed on the food or crop. As an example, and not by way of limitation, the user may select an additional test to be performed for a particular food or crop. In particular embodiments, the computing device of testing system 102 may be programmed to drive the touch screen UI terminal 110 to present the results of the testing performed on a food or crop.

In particular embodiments, the touch screen UI terminal 110 may be physically coupled or wirelessly coupled to the testing system 102. As an example, and not by way of limitation, the touch screen UI terminal 110 may comprise a display device that is physically coupled to the testing system 102. As another example and not by way of limitation, the touch screen UI terminal 110 may comprise a mobile computing device that is physically separate from the testing system 102 and that wirelessly communicates with the testing system. A cab computer of a tractor or other vehicle may be used. The mobile computing device may have an installed application or app compatible with and interoperating with mobile testing system 102 using app-based application-level network communication protocols that allow the user to interface the testing system 102 via the mobile computing device. In particular embodiments, the computing device of testing system 102 may be programmed to drive the touch screen UI terminal 110 to present instructions for the user to follow to test a food or crop. As an example, and not by way of limitation, the computing device of testing system 102 may be programmed to drive the touch screen UI terminal 110 to provide instructions for the user to supply a certain amount of a food or crop to perform testing on the food or crop.

In particular embodiments, the wireless database connection 112 may be one or more of a cellular connection, WiFi connection, Bluetooth or other near-field radio-frequency communication link to connect to the testing database 116 via the network 114. The network 114 broadly represents one more of an ad hoc network, an intranet, an extranet, a virtual private network (VPN), a local area network (LAN), a wireless LAN (WLAN), a wide area network (WAN), a wireless WAN (WWAN), a metropolitan area network (MAN), an internetwork, a portion of the Public Switched Telephone Network (PSTN), a cellular telephone network, or a combination of two or more of these.

In particular embodiments, the testing database 116 may be programmed to store data that is organized according to specific data structures, for example, as a relational, columnar, correlation, or object database. In particular embodiments, the testing database 116 may receive data from a plurality of mobile testing systems 102. The testing database 116 may process the data to generate baseline or controlled testing data for each food or crop. The testing database 116 may store scientific values for each food or crop based on raw received testing data such as output values from tester 108. In particular embodiments, the testing database 116 also may be programmed to generate human readable scores based on the testing data. As an example, and not by way of limitation, the data received from a testing system from a refractometer may be converted into a human readable score, such as a sweetness level of a tested food or crop.

In particular embodiments, the testing database 116 may establish a baseline or controlled testing data for a particular food or crop based on a particular set of received testing data. As an example, and not by way of limitation, an initial round of testing of a particular food or crop may be done in a controlled environment to generate the baseline or controlled testing data for the food or crop. In particular embodiments, the testing database 116 may receive data corresponding to outside data sources. As an example, and not by way of limitation, the consumer may indicate whether a particular food or crop is sweet. The testing database 116 may account for the consumer qualitative data by logging the consumer feedback.

In particular embodiments, the testing database 116 may generate a taste archetype based on the testing data from various sources corresponding to a food or crop. In this context, a taste archetype comprises a collection of ideal taste category scores based upon laboratory-derived baselines, expert tasters, and/or consumer data processed using machine learning algorithms. As an example, and not by way of limitation, the testing database 116 may compile data and generate a taste archetype for oranges, which would have specific physical and chemical properties as compared to other food or crops. In particular embodiments, the testing database 116 may compile data and generate a sub taste archetype based on the testing data. Furthermore, the testing database 116 may be programmed to derive, from a taste archetype, a human-readable varietal overall score value. Each human-readable varietal overall score value does not have a linear relationship to any known descriptions or values for taste attributes.

Different scaling factors for transforming tested scientific values into taste scores may be used for different foods, varieties, or varietals. As one example, TABLE 1 shows a possible mapping of output taste score values to scientific values or raw tested values for acid, Brix, ratio, weight, and other attributes for Valencia oranges:

TABLE 1

| | Score | | | | | | | | | |
|---|---|---|---|---|---|---|---|---|---|---|
| | 1 | 2 | 3 | 4 | 5 | 6 | 7 | 8 | 9 | 10 |
| Acid | 0 | 0.1 | 0.2 | 0.3 | 0.4 | 0.45 | 0.5 | 0.55 | 0.6 | >.7 |
| Brix | <4 | 5 | 6 | 7 | 8 | 9 | 10 | 11 | 12 | >12 |
| Ratio | <8 | 8 | 10 | 12 | 14 | 16 | 18 | 20 | 25 | >25 |
| Weight | <5 | 6 | 7 | 8 | 9 | 10 | 11 | 12 | 13 | >13 |
| Size | NA | | | | | | | | | |
| Color | NA | | | | | | | | | |

TABLE 1 may be stored in database 116 to form a data-driven basis for programmed transformations of scientific values from testing a substance, such as Valencia orange juice, using tests such as acid, Brix, ratio, and weight, into output taste score values that are suitable for publication or consumption by consumers. For example, if multispectral tester 108 yields an acid value of 0.55 as raw output from a test of Valencia orange juice, that value may be transformed to a score value of “8”. If the same juice sample has a tested Brix value of “10” as raw output, the Brix value would transform to a score value of “7”. A final consumer score value could be derived as “7.5”, being the arithmetic mean of “7” and “8”, or scaled or weighted to another value such as “7.8,” if one attribute is known to have greater influence on human taste perception of Valencia oranges than another attribute.

As another example, TABLE 2 shows a possible mapping of output taste score values to scientific values or raw tested values for acid, Brix, ratio, weight, and other attributes for Hamlin oranges:

TABLE 2

| | Score | | | | | | | | | |
|---|---|---|---|---|---|---|---|---|---|---|
| | 1 | 2 | 3 | 4 | 5 | 6 | 7 | 8 | 9 | 10 |
| Acid | 0 | 0.1 | 0.2 | 0.3 | 0.35 | 0.4 | 0.45 | 0.5 | 0.55 | >.5 |
| Brix | <1 | 2 | 3 | 4 | 5 | 6 | 7 | 8 | 9 | >10 |
| Ratio | <8 | 8 | 10 | 12 | 14 | 16 | 18 | 20 | 25 | >25 |
| Weight | <4 | 5 | 6 | 7 | 8 | 9 | 10 | 11 | 12 | >12 |
| Size | NA | | | | | | | | | |
| Color | NA | | | | | | | | | |

TABLE 3 illustrates example results of mapping values for a particular test of a sample Valencia orange:

TABLE 3
Sample 1 (Valencia)

| Conventional | Scientific | Consumer | Consumer |
|---|---|---|---|
| Attribute | Value | Attribute | Score |
| Acid | 0.46 | Tart/Bitter | 6.1 |
| Brix | 11.5 | Sweet | 8.4 |
| Ratio | 25 | Balance | 9.2 |
| Weight | 10.9 | Weight | 6.5 |
| Size | NA | Size | 6.2 |
| Color | No. 1 | Color | 7.3 |

| | |
|---|---|
| Overall Consumer Score | 7.20 |

TABLE 4 illustrates example results of mapping values for a particular test of a sample Hamlin orange:

TABLE 4
Sample 2 (Hamlin)

| Conventional | Scientific | Consumer | Consumer |
|---|---|---|---|
| Attribute | Value | Attribute | Score |
| Acid | 0.55 | Tart/Bitter | 4.2 |
| Brix | 8.1 | Sweet | 8.1 |
| Ratio | 20.18 | Balance | 6.9 |
| Weight | 9.8 | Weight | 6.3 |
| Size | NA | Size | 5.5 |
| Color | Fancy | Color | 9.1 |

| | |
|---|---|
| Overall Consumer Score | 6.68 |

In particular embodiments, the testing database 116 may be programmed to generate a label for each food or crop as described herein. The label may indicate human readable scores calculated for the particular food or crop. As an example, and not by way of limitation, the testing database 116 may generate a label for an orange to indicate the sweetness of the orange is “7” from a scale of “1” to “10.” As another example and not by way of limitation, the label for an orange may indicate the sweetness of the orange is in the top 80^{th} percentile.

In particular embodiments, the testing system 102 may receive label data from the testing database 116. The testing system 102 may be coupled to a label printer and may be programmed to print the label to be placed on the food or crop and/or a container of the food or crop. As an example, and not by way of limitation, the testing system 102 may be programmed to receive label data comprising a name or varietal, description, and unique identifier, and to print a label to attach to a food item, box, crate, or other carrier or package. The unique identifier may be encoded in the label data, alone or with other data such as a URL of a web page, as a QR code, barcode, or other encoded visual indicia. The unique identifier may comprise a rowid of a record in database 116 that contains testing data and score values for a specified food item, or a globally unique identifier (GUID) that the testing system 102 generates and assigns in response to initiation of testing and stores in a column of the database. In this manner, the unique identifier may be used to retrieve a record of data later or may be associated with a web page of another system that is programmed to display test data to remote users.

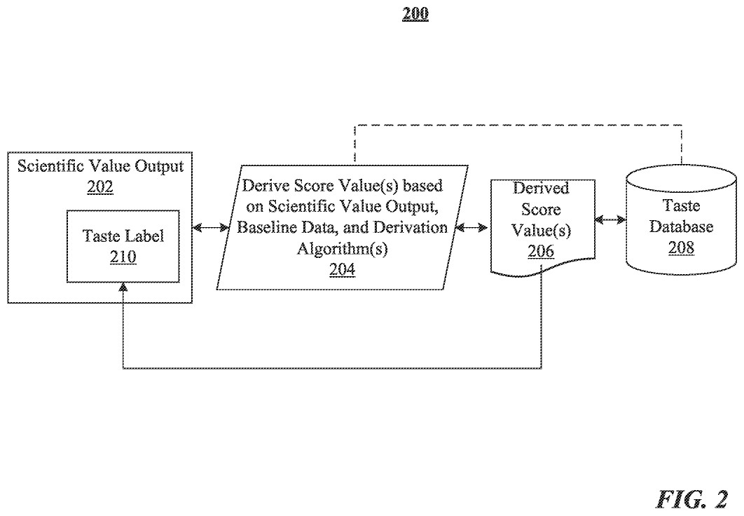

FIG. 2 illustrates an example process for deriving score values for a tested food or crop. FIG. 2 and each other flow diagram herein is intended as an illustration at the functional level at which skilled persons, in the art to which this disclosure pertains, communicate with one another to describe and implement algorithms using programming. The flow diagrams are not intended to illustrate every instruction, method object or sub-step that would be needed to program every aspect of a working program, but are provided at the same functional level of illustration that is normally used at the high level of skill in this art to communicate the basis of developing working programs.

In FIG. 2, a process 200 initiates using scientific value output 202. As described herein, a mobile testing system 100 may generate testing data which may be used to generate the scientific value output 202 comprising raw or unmodified values from testing devices. As an example, and not by way of limitation, the mobile testing system 100 may use the multispectral tester 108 to generate scientific value output 202. The scientific value output 202 may be compiled of testing data for a food or crop from a testing procedure using a plurality of testing devices.

The process 200 may continue with a calculation step 204 by using the scientific value output 202, baseline data, and one or more derivation algorithms to derive score values 206. As shown in FIG. 2, the calculation step 204 receives data from a taste database 208 to derive the score values 206. The calculation step 204 may receive baseline data from the taste database 208. In particular embodiments, the taste database 208 may be the same as the testing database 116, or may comprise a set of tables in the database 116, or a separate database.

In particular embodiments, the derivation algorithms may include one or more machine learning models to transform the scientific value output based on the baseline data to the derived score values 206. In particular embodiments, the derived score values 206 may indicate values corresponding to one or more of sugar, acidity, aromas, texture, color, weight, temperature, other gaseous properties, chemical breakdown such as freshness stability, and physical breakdown such as ripeness stability.

The process 200 may take the derived score values 206 to generate a taste label 210 that is associated with the tested food or crop corresponding to the scientific value output 202. The taste label 210 may refer to the derived score values 206, which may be human readable scores corresponding to the tested food or crop. As an example, and not by way of limitation, the taste label 210 may include a QR code, barcode, or other indicia capable of scanning with a mobile computing device to access human readable scores of the food or crop, such as sweetness level, acidity level, etc. of the food or crop. For example, in some embodiments, a grower may print the taste label 210 in the field after conducting mobile sampling, and apply the taste label to a crate, carton, or other packaging. A retailer may display the taste label 210 at the point of sale, enabling a consumer to use a mobile computing device to scan the taste label and retrieve a web page or data in a mobile app to display score values. Or, the retailer may scan the taste label before displaying the associated food items to consumers and generate other labels, signs, or displays, in a format that the retailer prefers, and then display the retailer's own labels, signs, or displays with the food item at the point of offering or point of sale.

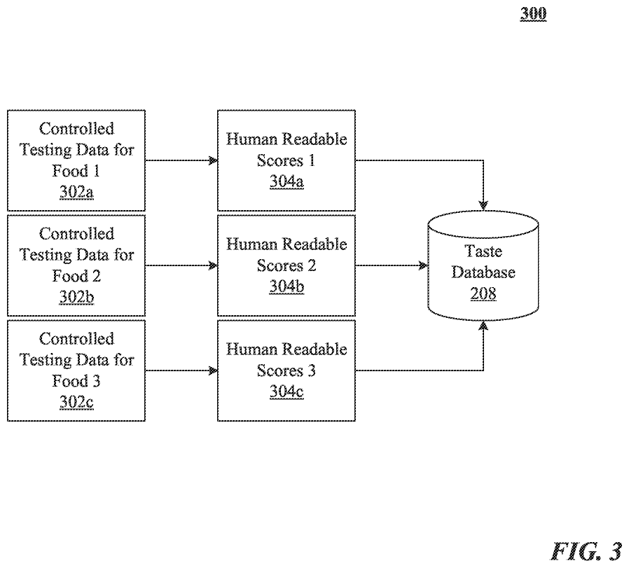

FIG. 3 illustrates an example process for compiling data for a taste database. In particular embodiments, a process 300 may start with receiving controlled testing data for food 302, which may be used to generate human readable scores 304. The human readable scores 304 may then be stored into the taste database 208 (FIG. 2). The controlled testing data for food 302 may also be stored with the human readable scores 304.

As an example, and not by way of limitation, the controlled testing data for food 1 302a may be stored in the taste database 208 with human readable scores 1 304a. This associates the controlled testing data for food 1 302a to the human readable scores 1 304a. This similarly applies to the controlled testing data for food 2 302b and human readable scores 2 304b pair and the controlled testing data for food 3 302c and human readable scores 3 304c pair. As described herein, the mobile testing system 100 may access the taste database 208 to retrieve baseline data for a food or crop. If the controlled testing data for food 1 302a corresponds to the requested baseline data, the taste database 208 may send baseline data comprising one or more of the controlled testing data for food 1 302a, human readable scores 1 304a, a derivation of one or more of these values, or a combination of these values. The mobile testing system 100 may use the baseline data to generate human readable scores for testing data as described herein.

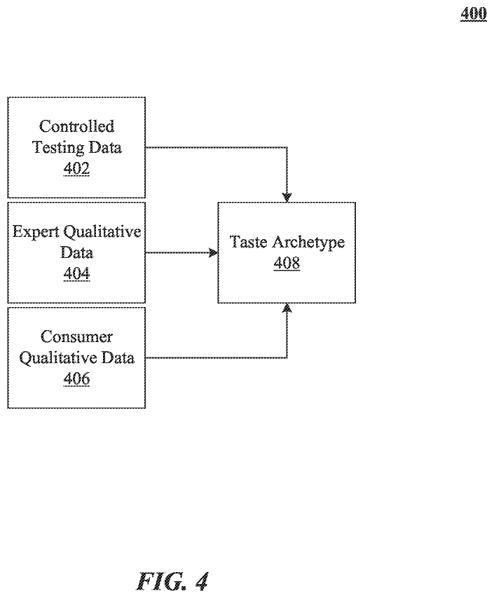

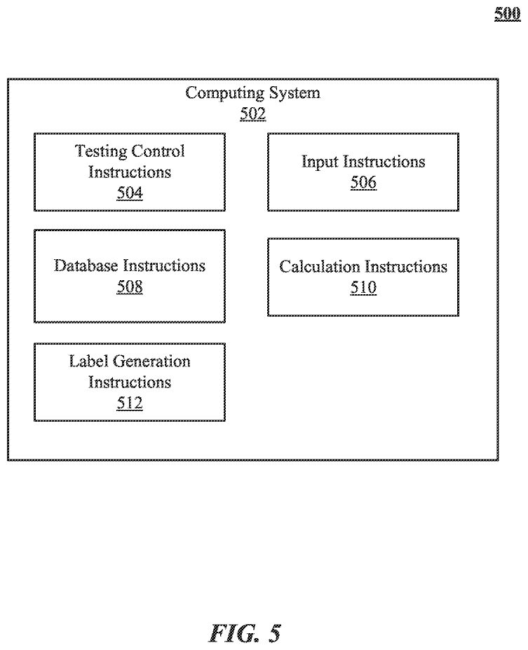

FIG. 4 illustrates an example process for generating a taste archetype. In particular embodiments, a process 400 may compile controlled testing data 402, expert qualitative data 404, and consumer qualitative data 406 to generate a taste archetype 408 for a food or crop. A taste archetype 408 may be a collective of values that indicate what a food or crop may taste, feel, and look like.

As an example, and not by way of limitation, a taste archetype 408 may indicate a sweetness for a food or crop. For instance, an orange taste archetype may indicate a sweetness of 8/10. In particular embodiments, the taste archetype 408 may also include subtypes for varieties of a food or crop. As an example, and not by way of limitation, an orange taste archetype may include a Valencia orange taste archetype and Cara Cara orange taste archetype that each have a particular set of values.

In particular embodiments, the controlled testing data 402 may be data collected from a user in a controlled environment corresponding to a food or crop. In particular embodiments, the controlled testing data 402 may include data captured from the field. The controlled testing data 402 may include one or more values captured from a multispectral tester 108 as described herein. In particular embodiments, the expert qualitative data 404 may include feedback and data from chefs, food scientists, and other food professionals. In particular embodiments, the consumer qualitative data 406 may include consumer feedback for a food or crop.

As described herein, a label may be generated for a food or crop of a particular taste archetype. Chefs, food scientists, and consumers alike may provide feedback for a food or crop by using the label. In particular embodiments, the label may be a link that a person may access to provide feedback. As an example, and not by way of limitation, a consumer may indicate that an orange that they ate was sweeter than normal oranges of the taste archetype. The taste archetype 408 may be updated based on the continual incoming data from experts and consumers and the occasional updated controlled testing data 402.

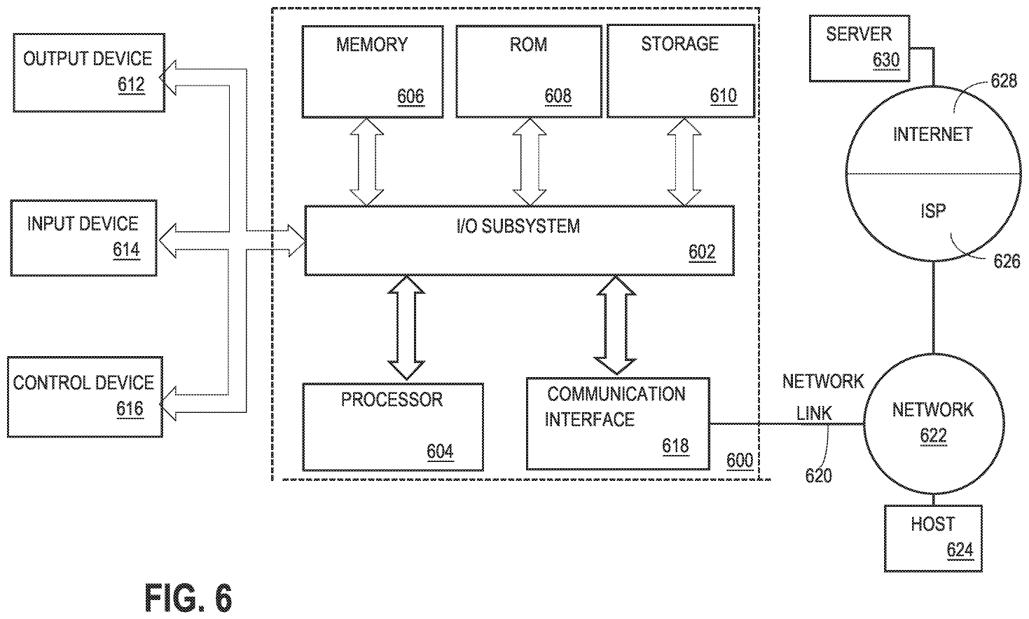

FIG. 5 illustrates a computing system to perform one or more functions described herein. In a computing environment 500, such as a virtual computing center or datacenter, a computing system 502 may include testing control instructions 504, input instructions 506, database instructions 508, calculation instructions 510, and label generation instructions 512. While certain components of computing system 502 are shown, the computing system 502 may have a different arrangement of components. As an example, and not by way of limitation, the computing system 502 may include further instructions. As another example and not by way of limitation, the computing system 502 may combine the testing control instructions 504 and input instructions 506.

In particular embodiments, the testing control instructions 504 may be used to perform testing on a food or crop as described herein. For a specific food or crop, the testing control instructions 506 may be configured to perform certain tests. As an example, and not by way of limitation, for testing an orange, testing using a refractometer, mass spectrometer, IR camera, and a digital scale may be used. As another example and not by way of limitation, for testing strawberries, testing using a mass spectrometer, IR camera, digital scale, and thermometer may be used. In particular embodiments, the testing control instructions 504 may receive data from input instructions 506. As an example, and not by way of limitation, input instructions 506 may send data indicating what food or crop is being tested. Testing control instructions 504 may communicate with other components of the computing system 502. Testing control instructions 504 may receive the data from input instructions 506 and calibrate testing systems to perform testing for the indicated food or crop. Testing control instructions 504 may receive data from performing testing on a food or crop. In particular embodiments, the testing control instructions 504 may send the testing data to other components of the computing system 502. As an example, and not by way of limitation, the testing control instructions 504 may send the data derived from tests performed on the food or crop (e.g., the raw data values or the scientific data values) to the calculation instructions 510 to derive scores (e.g., the human readable scores).

In particular embodiments, the input instructions 506 may be used to receive inputs from the user/tester. The input instructions 506 may interact with a touch screen UI terminal to receive inputs. As an example, and not by way of limitation, a user may select what food or crop is being tested through a touch screen UI terminal. The input instructions 506 may receive the selection and send the information to other components of computing system 502. As an example, and not by way of limitation, the input instructions 506 may send the information to testing control instructions 504 to calibrate the system to perform a test for a selected food or crop. As another example and not by way of limitation, the input instructions 506 may send the information to database instructions 508 to identify the selected food or crop to be used to access baseline data.

In particular embodiments, the database instructions 508 may be used to access a database for deriving scores. In particular embodiments, the database instructions 508 may receive information from input instructions 506 of a selected food or crop that is being tested. The database instructions 508 may access a database (e.g., taste database 208) to retrieve baseline data for the selected food or crop that is being tested. In particular embodiments, the database instructions 508 may send data to other components of the computing system 502. As an example, and not by way of limitation, the database instructions 508 may send data to the calculation instructions 510 to calculate derived scores.

In particular embodiments, the calculation instructions 510 may perform calculations to generate derived scores, human readable scores, and other scores as described herein. In particular embodiments, calculation instructions 510 may receive testing data for a food or crop from testing control instructions 504 and a baseline data for that food or crop from database instructions 508. In particular embodiments, the calculation instructions 510 may use a machine-learning model to generate derived scores from the testing data and the baseline data. The derived scores may be indicative of human readable scores for the tested food or crop. As an example, and not by way of limitation, the derived scores may indicate a sweetness level of the food or crop. In particular embodiments, the calculation instructions 510 may send the derived scores to other components of the computing system 502. As an example, and not by way of limitation, the calculation instructions 510 may send the derived scores to the database instructions 508 to upload to the database. As another example and not by way of limitation, the derived scores may be sent to the label generation instructions 512 to generate a label.

In particular embodiments, the label generation instructions 512 may receive the derived scores from the calculation instructions 510. In particular embodiments, the label generation instructions 512 may receive a label from a database via database instructions 508. In particular embodiments, the label generation instructions 512 may generate a label (e.g., a QR code) based on the derived scores received from the calculation instructions 510.

In particular embodiments, the derived scores and label may be sent to a third-party server to process. In particular embodiments, the third-party server may host a marketplace website where the derived scores and label may be added for particular products. As an example, and not by way of limitation, after a food or crop is tested out in the field, a label is generated that is attached to the food or crop. For instance, a sticker may be applied to a pallet of blueberries. The blueberries from the pallet may subsequently be packed in containers that may also include the sticker. Once the pack of blueberries arrive in a grocery store to be sold to an end consumer, the consumer may interact with the label to access the derived scores of the food or crop. In particular embodiments, a third-party server may process the derived scores and label to regenerate a label with a subset of the derived scores. As an example, and not by way of limitation, if an entity associated with the third-party server determines a consumer is typically only interested with a sweetness level and an acidity level of an orange, then the entity may remove other derived scores and only include the derived scores corresponding to the sweetness level and acidity level in a label, which the consumer may access. In particular embodiments, the marketplace website may include the derived scores for a product page. As an example, and not by way of limitation, continuing the blueberry example, the marketplace website may have a container containing the blueberry product with details corresponding to the product. The details may contain information (e.g., weight) of the product, but also include the derived scores that the consumer may be interested to see.

### 6. Implementation Example—Hardware Overview

According to one embodiment, the techniques described herein are implemented by at least one computing device. The techniques may be implemented in whole or in part using a combination of at least one server computer and/or other computing devices that are coupled using a network, such as a packet data network. The computing devices may be hard-wired to perform the techniques, or may include digital electronic devices such as at least one application-specific integrated circuit (ASIC) or field programmable gate array (FPGA) that is persistently programmed to perform the techniques, or may include at least one general purpose hardware processor programmed to perform the techniques pursuant to program instructions in firmware, memory, other storage, or a combination. Such computing devices may also combine custom hard-wired logic, ASICs, or FPGAs with custom programming to accomplish the described techniques. The computing devices may be server computers, workstations, personal computers, portable computer systems, handheld devices, mobile computing devices, wearable devices, body mounted or implantable devices, smartphones, smart appliances, internetworking devices, autonomous or semi-autonomous devices such as robots or unmanned ground or aerial vehicles, any other electronic device that incorporates hard-wired and/or program logic to implement the described techniques, one or more virtual computing machines or instances in a data center, and/or a network of server computers and/or personal computers.

FIG. 6 is a block diagram that illustrates an example computer system with which an embodiment may be implemented. In the example of FIG. 6, a computer system 600 and instructions for implementing the disclosed technologies in hardware, software, or a combination of hardware and software, are represented schematically, for example as boxes and circles, at the same level of detail that is commonly used by persons of ordinary skill in the art to which this disclosure pertains for communicating about computer architecture and computer systems implementations.

Computer system 600 includes an input/output (I/O) subsystem 602 which may include a bus and/or other communication mechanism(s) for communicating information and/or instructions between the components of the computer system 600 over electronic signal paths. The I/O subsystem 602 may include an I/O controller, a memory controller and at least one I/O port. The electronic signal paths are represented schematically in the drawings, for example as lines, unidirectional arrows, or bidirectional arrows.

At least one hardware processor 604 is coupled to I/O subsystem 602 for processing information and instructions. Hardware processor 604 may include, for example, a general-purpose microprocessor or microcontroller and/or a special-purpose microprocessor such as an embedded system or a graphics processing unit (GPU) or a digital signal processor or ARM processor. Processor 604 may comprise an integrated arithmetic logic unit (ALU) or may be coupled to a separate ALU.

Computer system 600 includes one or more units of memory 606, such as a main memory, which is coupled to I/O subsystem 602 for electronically digitally storing data and instructions to be executed by processor 604. Memory 606 may include volatile memory such as various forms of random-access memory (RAM) or other dynamic storage device. Memory 606 also may be used for storing temporary variables or other intermediate information during execution of instructions to be executed by processor 604. Such instructions, when stored in non-transitory computer-readable storage media accessible to processor 604, can render computer system 600 into a special-purpose machine that is customized to perform the operations specified in the instructions.

Computer system 600 further includes non-volatile memory such as read only memory (ROM) 608 or other static storage device coupled to I/O subsystem 602 for storing information and instructions for processor 604. The ROM 608 may include various forms of programmable ROM (PROM) such as erasable PROM (EPROM) or electrically erasable PROM (EEPROM). A unit of persistent storage 610 may include various forms of non-volatile RAM (NVRAM), such as FLASH memory, or solid-state storage, magnetic disk or optical disk such as CD-ROM or DVD-ROM and may be coupled to I/O subsystem 602 for storing information and instructions. Storage 610 is an example of a non-transitory computer-readable medium that may be used to store instructions and data which when executed by the processor 604 cause performing computer-implemented methods to execute the techniques herein.

The instructions in memory 606, ROM 608 or storage 610 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. The instructions may implement a web server, web application server or web client. The instructions may be organized as a presentation layer, application layer and data storage layer such as a relational database system using structured query language (SQL) or no SQL, an object store, a graph database, a flat file system or other data storage.

Computer system 600 may be coupled via I/O subsystem 602 to at least one output device 612. In one embodiment, output device 612 is a digital computer display. Examples of a display that may be used in various embodiments include a touch screen display or a light-emitting diode (LED) display or a liquid crystal display (LCD) or an e-paper display. Computer system 600 may include other type(s) of output devices 612, alternatively or in addition to a display device. Examples of other output devices 612 include printers, ticket printers, plotters, projectors, sound cards or video cards, speakers, buzzers or piezoelectric devices or other audible devices, lamps or LED or LCD indicators, haptic devices, actuators or servos.

At least one input device 614 is coupled to I/O subsystem 602 for communicating signals, data, command selections or gestures to processor 604. Examples of input devices 614 include touch screens, microphones, still and video digital cameras, alphanumeric and other keys, keypads, keyboards, graphics tablets, image scanners, joysticks, clocks, switches, buttons, dials, slides, and/or various types of sensors such as force sensors, motion sensors, heat sensors, accelerometers, gyroscopes, and inertial measurement unit (IMU) sensors and/or various types of transceivers such as wireless, such as cellular or Wi-Fi, radio frequency (RF) or infrared (IR) transceivers and Global Positioning System (GPS) transceivers.

Another type of input device is a control device 616, which may perform cursor control or other automated control functions such as navigation in a graphical interface on a display screen, alternatively or in addition to input functions. Control device 616 may be a touchpad, a mouse, a trackball, or cursor direction keys for communicating direction information and command selections to processor 604 and for controlling cursor movement on display 612. The input device may have at least two degrees of freedom in two axes, a first axis (e.g., x) and a second axis (e.g., y), that allows the device to specify positions in a plane. Another type of input device is a wired, wireless, or optical control device such as a joystick, wand, console, steering wheel, pedal, gearshift mechanism or other type of control device. An input device 614 may include a combination of multiple different input devices, such as a video camera and a depth sensor.

In another embodiment, computer system 600 may comprise an internet of things (IoT) device in which one or more of the output device 612, input device 614, and control device 616 are omitted. Or, in such an embodiment, the input device 614 may comprise one or more cameras, motion detectors, thermometers, microphones, seismic detectors, other sensors or detectors, measurement devices or encoders and the output device 612 may comprise a special-purpose display such as a single-line LED or LCD display, one or more indicators, a display panel, a meter, a valve, a solenoid, an actuator or a servo.

When computer system 600 is a mobile computing device, input device 614 may comprise a global positioning system (GPS) receiver coupled to a GPS module that is capable of triangulating to a plurality of GPS satellites, determining and generating geo-location or position data such as latitude-longitude values for a geophysical location of the computer system 600. Output device 612 may include hardware, software, firmware and interfaces for generating position reporting packets, notifications, pulse or heartbeat signals, or other recurring data transmissions that specify a position of the computer system 600, alone or in combination with other application-specific data, directed toward host 624 or server 630.

Computer system 600 may implement the techniques described herein using customized hard-wired logic, at least one ASIC or FPGA, firmware and/or program instructions or logic which when loaded and used or executed in combination with the computer system causes or programs the computer system to operate as a special-purpose machine. According to one embodiment, the techniques herein are performed by computer system 600 in response to processor 604 executing at least one sequence of at least one instruction contained in main memory 606. Such instructions may be read into main memory 606 from another storage medium, such as storage 610. Execution of the sequences of instructions contained in main memory 606 causes processor 604 to perform the process steps described herein. In alternative embodiments, hard-wired circuitry may be used in place of or in combination with software instructions.

The term “storage media” as used herein refers to any non-transitory media that store data and/or instructions that cause a machine to operation in a specific fashion. Such storage media may comprise non-volatile media and/or volatile media. Non-volatile media includes, for example, optical or magnetic disks, such as storage 610. Volatile media includes dynamic memory, such as memory 606. Common forms of storage media include, for example, a hard disk, solid state drive, flash drive, magnetic data storage medium, any optical or physical data storage medium, memory chip, or the like.

Storage media is distinct from but may be used in conjunction with transmission media. Transmission media participates in transferring information between storage media. For example, transmission media includes coaxial cables, copper wire and fiber optics, including the wires that comprise a bus of I/O subsystem 602. Transmission media can also take the form of acoustic or light waves, such as those generated during radio-wave and infra-red data communications.

Various forms of media may be involved in carrying at least one sequence of at least one instruction to processor 604 for execution. For example, the instructions may initially be carried on a magnetic disk or solid-state drive of a remote computer. The remote computer can load the instructions into its dynamic memory and send the instructions over a communication link such as a fiber optic or coaxial cable or telephone line using a modem. A modem or router local to computer system 600 can receive the data on the communication link and convert the data to a format that can be read by computer system 600. For instance, a receiver such as a radio frequency antenna or an infrared detector can receive the data carried in a wireless or optical signal and appropriate circuitry can provide the data to I/O subsystem 602 such as place the data on a bus. I/O subsystem 602 carries the data to memory 606, from which processor 604 retrieves and executes the instructions. The instructions received by memory 606 may optionally be stored on storage 610 either before or after execution by processor 604.

Computer system 600 also includes a communication interface 618 coupled to bus 602. Communication interface 618 provides a two-way data communication coupling to network link(s) 620 that are directly or indirectly connected to at least one communication networks, such as a network 622 or a public or private cloud on the Internet. For example, communication interface 618 may be an Ethernet networking interface, integrated-services digital network (ISDN) card, cable modem, satellite modem, or a modem to provide a data communication connection to a corresponding type of communications line, for example an Ethernet cable or a metal cable of any kind or a fiber-optic line or a telephone line. Network 622 broadly represents a local area network (LAN), wide-area network (WAN), campus network, internetwork or any combination thereof. Communication interface 618 may comprise a LAN card to provide a data communication connection to a compatible LAN, or a cellular radiotelephone interface that is wired to send or receive cellular data according to cellular radiotelephone wireless networking standards, or a satellite radio interface that is wired to send or receive digital data according to satellite wireless networking standards. In any such implementation, communication interface 618 sends and receives electrical, electromagnetic or optical signals over signal paths that carry digital data streams representing various types of information.

Network link 620 typically provides electrical, electromagnetic, or optical data communication directly or through at least one network to other data devices, using, for example, satellite, cellular, Wi-Fi, or BLUETOOTH technology. For example, network link 620 may provide a connection through a network 622 to a host computer 624.

Furthermore, network link 620 may provide a connection through network 622 or to other computing devices via internetworking devices and/or computers that are operated by an Internet Service Provider (ISP) 626. ISP 626 provides data communication services through a world-wide packet data communication network represented as internet 628. A server computer 630 may be coupled to internet 628. Server 630 broadly represents any computer, data center, virtual machine or virtual computing instance with or without a hypervisor, or computer executing a containerized program system such as DOCKER or KUBERNETES. Server 630 may represent an electronic digital service that is implemented using more than one computer or instance and that is accessed and used by transmitting web services requests, uniform resource locator (URL) strings with parameters in HTTP payloads, API calls, app services calls, or other service calls. Computer system 600 and server 630 may form elements of a distributed computing system that includes other computers, a processing cluster, server farm or other organization of computers that cooperate to perform tasks or execute applications or services. Server 630 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. Server 630 may comprise a web application server that hosts a presentation layer, application layer and data storage layer such as a relational database system using structured query language (SQL) or no SQL, an object store, a graph database, a flat file system or other data storage.

Computer system 600 can send messages and receive data and instructions, including program code, through the network(s), network link 620 and communication interface 618. In the Internet example, a server 630 might transmit a requested code for an application program through Internet 628, ISP 626, local network 622 and communication interface 618. The received code may be executed by processor 604 as it is received, and/or stored in storage 610, or other non-volatile storage for later execution.

The execution of instructions as described in this section may implement a process in the form of an instance of a computer program that is being executed and consisting of program code and its current activity. Depending on the operating system (OS), a process may be made up of multiple threads of execution that execute instructions concurrently. In this context, a computer program is a passive collection of instructions, while a process may be the actual execution of those instructions. Several processes may be associated with the same program; for example, opening up several instances of the same program often means more than one process is being executed. Multitasking may be implemented to allow multiple processes to share processor 604. While each processor 604 or core of the processor executes a single task at a time, computer system 600 may be programmed to implement multitasking to allow each processor to switch between tasks that are being executed without having to wait for each task to finish. In an embodiment, switches may be performed when tasks perform input/output operations, when a task indicates that it can be switched, or on hardware interrupts. Time-sharing may be implemented to allow fast response for interactive user applications by rapidly performing context switches to provide the appearance of concurrent execution of multiple processes simultaneously. In an embodiment, for security and reliability, an operating system may prevent direct communication between independent processes, providing strictly mediated and controlled inter-process communication functionality.

In the foregoing specification, embodiments of the invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

What is claimed is:

1. A mobile testing system, comprising:
a modular testing attachment comprising at least one of a crusher attachment, a squeezer attachment, and a blender attachment, the modular testing attachment configured to obtain a substance from food or crop products;
a universal attachment coupled to the modular testing attachment and structured to receive and advance the substance from the food or crop products;
a multispectral tester coupled to the universal attachment and structured to receive the substance from the universal attachment and test the substance to output scientific values; and
a computing system operably coupled to the modular testing attachment and the multispectral tester and configured to selectively or automatically operate components thereof

2. The system of claim 1, the multispectral tester comprising at least one of a refractometer, a mass spectrometer, an infrared camera, a visible light camera, a thermometer, modular bays, and a digital scale for mass measurements.

3. The system of claim 1, the universal attachment comprising a vacuum mechanism structured to advance the substance from the modular testing attachment to the multispectral tester.

4. The system of claim 1 the universal attachment coupled to multiple testing attachments and the computing system configured to determine a type of food or crop product that is being tested, and automatically select and activate one of the multiple testing attachments that correspond to the type of food or crop product that is being tested.

5. The system of claim 1, the computing system configured to access baseline data from a database to process testing data for the substance and scale or weight the scientific values based on the baseline data.

6. The system of claim 5, further comprising a network interface coupled to the computing system, and the computing system configured to generate human-readable scores based on the scientific values, and store the human-readable scores and the scientific values within the database.

7. The system of claim 6, the human-readable scores related to at least one of sweetness, balance, weight, size, color, and bitterness.

8. The system of claim 1, further comprising a fluid tank structured to provide a fluid comprising at least one of distilled water, isopropyl alcohol, and disinfectant to flush out the multispectral tester automatically after testing the substance.

9. The system of claim 1, further comprising two or more multispectral testers, each structured to receive at least a portion of the substance from the universal attachment, the computing system configured to determine a type of food or product being tested and selectively activate one or more of the multispectral testers based on the type of food or product being tested.

10. The system of claim 9, further comprising a user-interface screen coupled to the computing system configured to allow a user to select the type of food or product being tested.

11. A computer-implemented method of testing food items, the method comprising:
obtaining scientific data values for one or more conventional attributes of a food item;
transforming each of the scientific data values into two or more consumer score values using a digitally stored scaled mapping of scientific data values to consumer score values;
forming a final consumer score value by combining and weighting the two or more consumer score values, and
outputting the final consumer score value, wherein the final consumer score value represents a taste archetype for the food item.

12. The method of claim 11, further comprising generating label data comprising an encoded representation of the final consumer score value and printing a label comprising the encoded representation of the final consumer score value.

13. The method of claim 11, further comprising obtaining the scientific values by activating a mobile testing system structured to receive the food item, process the food item, and test the food item based on a type of the food item.

14. The method of claim 13, further comprising:
receiving, via a user interface, a selection of the type of the food item;
activating one or more modular testing attachments of the mobile testing system, the one or more modular testing attachments structured to obtain a substance from the food item; and
generating the scientific data values using one or more multispectral tester devices that receive the substance.

15. The method of claim 11, the scientific data values representing scores related to at least one of a pH scale, a Brix scale, a weight, and a color of the food item.

16. The method of claim 11, the consumer scale values indicate at least one of sweetness, balance, weight, size, color, and bitterness.

17. A system comprising:
a mobile testing system configured to receive a food item and generate scientific data values for one or more conventional attributes of the food item, the mobile testing system comprising:
a modular testing attachment structured to obtain a substance from the food item; and
a multispectral tester structured to receive the substance and test the substance to generate scientific data values;
one or more processors coupled to the mobile testing system; and
a memory device coupled to one or more processors, the memory device storing instructions that, when executed by the one or more processors, cause the operations of:
obtaining scientific data values from the mobile testing system;
transforming each of the scientific data values into two or more consumer score values using a digitally stored scaled mapping of scientific data values to consumer score values;
forming a final consumer score value by combining and weighting the two or more consumer score values, and
outputting the final consumer score value, wherein the final consumer score value represents a physical characteristic of the food item.

18. The system of claim 17, the operations further comprising: generating label data comprising an encoded representation of the final consumer score value and printing a label comprising the encoded representation of the final consumer score value.

19. The system of claim 17, the scaled mapping of scientific data values to consumer score values being dependent on a type of the food item.

20. The system of claim 17, further comprising a user interface coupled to the one or more processors, the user interface configured to provide a plurality of selections to a user, each of the plurality of selections being related to a type of food items.
